(19) United States
(12) Patent Application Publication
(10) Pub. No.: US 2004/0231681 A1
(43) Pub. Date: Nov. 25, 2004

(54) Method of improving the emotional development of an infant

(76) Inventor: Derek John Goldsmith, Edenbridge (GB)

Correspondence Address: MCNAIR LAW FIRM, PA, P.O. BOX 10827, GREENVILLE, SC, 29603-0827, US

(21) Appl. No.: 10/849,362

(22) Filed: May 19, 2004

(30) Foreign Application Priority Data
May 23, 2003 (GB) 0311982.3

(51) Int. Cl. A61B 19/00

(52) U.S. Cl. 128/898; 600/28; 607/45

(57) ABSTRACT

The method of the present invention proposes to induce emotions of happiness or joy in a pregnant mother, with the intended result that the pleasant emotions can then be transmitted to the foetus through the normal mother-foetus communication.

### FIELD OF THE INVENTION

The present invention relates to improving the development of the mental state of an infant, for example to reduce the susceptibility of the infant to developing psychological disorders such as schizophrenia.

### PRIOR ART

In my U.S. Pat. No. 6,058,939 it is proposed that an infant be caused to have its sleep pattern controlled in order to ensure that as much as possible of the sleep experienced by the infant is rapid eye motion (REM) sleep. The method there proposed involved waking the infant when it had not entered REM sleep and then soothing the wakened infant to allow it to fall asleep once more, in a relaxed state more likely to lead to REM sleep.

The method of the above US patent was based on the belief that in REM sleep the emotional development of the infant is better than in non-REM sleep when the infant is in what I there termed a>frozen=state, reminiscent of a particularly helpful reflex in the rest of the animal kingdom and in particular in primitive man, where the infant would remain silent and motionless in order to reduce the likelihood of interference by a predator or other threat. I believe that the more healthy emotional development resulting from a higher proportion of sleep being in the REM phase could reduce the likelihood of psychosis developing in the pubescent and post-puberty stages of growth.

Surprisingly, it has been postulated that during pregnancy a foetus also has the propensity to undergo both REM and non-REM sleep. I therefore believe that insufficient REM sleep during foetal development is equally likely to contribute to a likelihood of mental problems in the growing infant and subsequent adult.

### OBJECTS OF THE INVENTION

Accordingly, it is a first object of the present invention to provide a method of improving the emotional development of an infant comprising causing a pregnant mother to experience the emotion of happiness during pregnancy, wherein the foetus carried by said pregnant mother becomes, after birth, said infant.

It is a second object of the invention to provide a method of improving the emotional development of an infant comprising exposing the foetus to signs of happiness during pregnancy, wherein the foetus becomes, after birth, said infant.

It is another object of the present invention to improve the emotional development of the foetus by transmitting sounds of laughter into the womb especially where this does not occur naturally.

The term Ajoy@ is often used to refer to the emotion herein described as Ahappiness@.

### DETAILED DESCRIPTION OF THE INVENTION

Modern humans have inherited a fear system that is far too powerful for modern civilized living and, without laughter to either wake them or transfer them into normal REM sleep, infants can be left in a>frozen=state. Infants can be>frozen=by seeing fear on a parent's face, or by otherwise seeing a similar facial expression.

Fear is also transmitted by a mother in the form of tension or shivering and/or by transmission of chemicals such as hormones, and this is detected by the foetus. It is now known that the amygdala plays an important part in this process and that its development begins in mid pregnancy. If this development hasn't taken place properly during pregnancy this deficit may impair the infant's ability to read its mother's face, and to sense mother's happiness as described above.

Early humans experienced fear on a daily basis. After the danger had passed the group would experience spontaneous and catching laughter that dispelled the fear-signalling hormones and tensions of fear. It has recently been discovered that the human amygdala responds differentially to both fear and happiness and/or laughter. It has been proposed by Morris, Ohman et al in 1998, that the right amygdala is the main brain region associated with the unconscious recognition of a negatively-charged face.

Logically there needs to be a relief from the tensions following periods of fear.

The tensions that a mother undergoes in modern times are not always followed by laughter and as a consequence the foetal amygdala may not develop properly, such that the newborn does not recognise mother's facial expressions particularly that of happiness.

This has serious implications for the mental health of the child. Because the natural transmission and stimulation role of emotions does not develop properly, so the important communication process does not occur.

Not until recently have the leading world experts begun to look at how important emotions are to our development early in life. Previously emotions have been thought of only as feelings, with little attention being given to how or why they arise. Yet emotions can be expressed on the adult face within milliseconds of their cause i.e. in a sudden road accident situation. Thus the face is acting as an extension of the brain.

Since Darwin's time scientists have sought to understand why and what is the relevance of facial expressions to other humans. The present application sets out to explain for the first time how important this role is to children relating to the new world that they are discovering, and to show the importance for children to read their parents' emotions correctly when they are so rapidly expressed directly in the context of the situations that give rise to them. Children typically do this out of the corner of their eye!

It is also known that the neonate can read its mother's face and, when surprised or startled, it focuses with eyes wide open on that face expecting to see fear.

Most importantly it is known that much mental illness has its roots in the incorrect interpretation of other people's emotions, particularly parental emotions.

Applying the rule that emotions transmit and stimulate, and that children read their parents' emotions in the context of the situations that give rise to them, we have the fundamental formulae for how children learn and relate to the world.

In Pavlov's terminology, the transmitted emotion is the natural or unconditioned stimulus (US) and the context, or the situation, that gave rise to the emotion is the conditioning stimulus (CS).

Thus, when the child is in the same contextual situation without its parents the context becomes the conditioning stimulus (CS) which produces the conditioned response (CR) in the child's brain and it experiences the same emotion, and in that emotion will recall how its parents dealt with that situation.

The pregnant mother may be subjected to a pleasant emotional stimulus by, for example, encouraging laughter. Methods of inducing a happy emotional state in the mother may include participation in a group activity involving discussions or other experiences, actions promoting laughter, or showing the mother amusing stimuli such as films, video recordings and drawings, or showing the mother amusing pieces of writing which will induce happiness and, preferably, laughter.

Another way of inducing happiness would be to transmit sounds of laughter into the womb, and this would be particularly effective in the case of the sound of female laughter, more especially the laughter of the mother in question. Thus mother's laughter could be recorded during a happy moment and she or another could then hold a speaker to her abdomen in order to cause the sounds of the laughter to pass through into the womb. This would be especially effective if carried out immediately after periods of tension, anxiety and fear if the mother does not naturally laugh at such times.

It is also expected that happiness can be induced by transmitting music to the foetus, preferably later in the pregnancy when the development is more advanced. Although music can also be transmitted during early pregnancy, it is envisaged that this will have the benefit of causing the pregnant mother to experience happiness which will then benefit the foetus, but it is known that a new born may respond to the rhythm of music which they learnt in the womb during the later stages of pregnancy, and that this can be associated with the emotion of happiness or joy. This is a known and desirable response, but the present invention is intended to be used in situations where the mother would not normally be in a situation where the foetus is exposed to music, and therefore the emotional development of the infant will be improved as compared with what would otherwise be expected. After birth, the new baby should be able to read its mother's facial happiness and respond with happiness, and it is envisaged that this response will be encouraged by exposing the foetus to music as well as other happiness—inducing phenomena.

The apparatus suitable for use in subjecting the foetus to happiness while still in the womb may comprise a flat loudspeaker, preferably having a suitable membrane or fluid-containing bag between the vibrating diaphragm and the exterior so that this membrane or bag can be placed in contact with the abdominal wall of the mother. Conventional sound recorders, power supplies and amplifiers will be capable of producing the desired signal to cause the loudspeaker to generate the desired sounds.

Each treatment session need only last for a few minutes in order to be effective, and the Atreatment@ may in itself improve the mood of the mother as well as benefiting the foetus. However, Aover dosing@ with such treatment will not be harmful. The treatment should be sufficient to educate the amygdala to respond to happiness and hence to differentiate from fear.

The frequency of treatment will depend on the state of well-being on the part of the mother; if she is depressive or negative it may be necessary to apply the treatment daily, but otherwise weekly sessions would be sufficient. However, this suggestion is in no way restrictive of the frequency which will be assessed by trial and error.

There has recently been discovered what has been called>The seasonality effect=whereby for both psychopathy and psychosis there is an increase of about 10% in the rate of occurrence for those born in springtime in both the northern hemisphere and the southern hemisphere, suggesting that the climatic changes are responsible (in view of the fact that Spring in the northern hemisphere will be six months displaced relative to Spring in the southern hemisphere). This is thought to be due to the less buoyant emotional state of mothers during the winter, and it is felt that the result is akin to the now well-known Seasonal Affective Disorder (SAD). This discovery is consistent with my proposal to encourage happy emotions on the part of a pregnant mother so as to avoid this seasonal variation in the tendency to psychosis and psychopathy. It is also a significant indicator to the need for laughter during pregnancy to promote the proper development of the foetal amygdala. The invention may therefore involve using any traditionally employed action to overcome the SAD phenomenon.

It is known that depression is a state of very low emotional activity, and that this may be the cause of some types of depression.

If the mother is subjected to happy emotions from at least mid-term of the pregnancy then the foetus may receive signals indicative of that pleasant emotional state, for example, through chemical (hormonal) transmission from the mother, or through other mechanical or acoustic stimuli. If the amygdala is thus educated to recognise laughter and hence to differentiate from fear, the natural response to happiness can be laid down in the foetal brain during development, such that these responses will function correctly after the birth of the baby.

Since the foetus does have the opportunity of entering either REM sleep or non-REM sleep, the likelihood of the sleep experienced by the foetus being REM sleep is improved if the mother can experience happy and positive emotion and transmit this to the offspring during pregnancy by the usual communication routes.

Another factor which is relevant to the proposal in accordance with the present invention is that it is known that there are various risk factors relating to the likelihood of schizophrenia, and these factors may be related to the family environment and history. For example, the likelihood of experiencing schizophrenia in later life is increased in the case of populations which have more recently become civilised, as compared with those having a longer history of civilised life. Recognition of the higher risk of mental disorders developing later may be effected by observing factors such as the presence of saccadic eye motion in the child, and detecting how readily how a child can read emotion (for example, by showing the child various facial expressions which range over a spectrum between happiness or joy and sadness to see which children more readily recognise the deviation from a neutral state). Given the fact that the likelihood of schizophrenia and other mental disorders is environment-related and family-related, it is now possible to identify those pregnant mothers whose children are more at risk, and to apply the method of the present invention more extensively in those cases, in order to reduce the likelihood of their offspring being vulnerable.

Given the possibility of brain imaging it may be possible to detect unusual conditions of the amygdala and this again may help to identify those cases where a higher vulnerability to mental disorders such as schizophrenia arises.

It is also believed to be possible to identify those females who are less able to read and transmit emotion than can the average adult, and are therefore likely to have offspring with a higher than average susceptibility to mental disorders such as schizophrenia. In such a case it will be particularly useful to apply the method of this invention.

Generally, it is felt that the child or adult who is better able to read emotion is a more healthy one, and the method of the present invention proposes to improve that ability by subjecting the pregnant mother to pleasant emotions with the intention that the pleasant emotions experienced by the mother will then be transferred to the foetus. The important feature of the present invention is that missing Ahappiness@ is replaced by either stimulating the mother to laugh or feeding signs of laughter directly to the foetus.

1. A method of improving the emotional development of an infant comprising causing a pregnant mother to experience happy emotions during pregnancy, wherein the foetus carried by said pregnant mother becomes, after birth, said infant.

2. A method according to claim 1, wherein the exposure of the mother to happy emotions is effected by inducing laughter in the mother as a result of exposure to amusing stimuli.

3. A method according to claim 1, and including using laughing gas, for example nitrous oxide, to induce happy emotions in the mother.

4. A method according to claim 1, wherein the experience of happy emotions is induced at least once per week.

5. A method according to claim 4, wherein the experience of happy emotions is induced daily.

6. A method according to claim 1, wherein the step of causing the mother and/or foetus to experience happy emotions begins at least at mid-term of the pregnancy.

7. A method according to claim 1, and including the step of identifying mothers whose offspring are more likely than average to be vulnerable to mental disorders such as schizophrenia, and selectively carrying out the method on those pregnant mothers.

8. A method according to claim 7, wherein the identification of mothers whose offspring are more vulnerable includes identifying those mothers who are less able to read and transmit emotion than can the average adult, and applying the method in the case of those mothers.

9. A method of improving the emotional development of an infant comprising exposing the foetus to signs of happiness during pregnancy, wherein the foetus becomes, after birth, said infant.

10. A method according to claim 9, wherein the step of exposing the foetus to signs of happiness comprises applying the sound of laughter to the womb in which the foetus is growing.

11. A method according to claim 10, wherein the application of the sound of laughter includes applying a sound transducer to the abdomen of the pregnant mother.

12. A method according to claim 9, wherein the laughter is female laughter.

13. A method according to claim 12, wherein the laughter is the sound of laughter of the pregnant mother.

14. A method according to claim 9, wherein the step of exposing the foetus to signs of happiness comprises applying the sound of music to the womb in which the foetus is growing.

15. A method according to claim 1, wherein the experience of happy emotions is induced at least once per week.

16. A method according to claim 4, wherein the experience of happy emotions is induced daily.

17. A method according to claim 1, wherein the step of causing the mother and/or foetus to experience happy emotions begins at least at mid-term of the pregnancy.
